(19) United States
(12) Patent Application Publication
(10) Pub. No.: US 2026/0140100 A1
(43) Pub. Date: May 21, 2026

(54) Breathalyzer Test Container Assembly For Storing And Releasing Gases

(71) Applicant: Francis J. O'Reagan, III, Dartmouth, MA (US)

(72) Inventor: Francis J. O'Reagan, III, Dartmouth, MA (US)

(21) Appl. No.: 19/120,943

(22) PCT Filed: Oct. 13, 2023
(86) PCT No.: PCT/US2023/076911

§ 371 Date: Apr. 14, 2025

Related U.S. Application Data
(60) Provisional application No. 63/416,077, filed on Oct. 14, 2022.

(51) Int. Cl. G01N 33/00; A61B 5/08; F17C 1/00; G01N 33/497

(52) U.S. Cl. CPC G01N 33/0072; F17C 1/00; A61B 5/082; F17C 2201/0104; F17C 2205/0323; F17C 2221/031; G01N 33/4972

(57) ABSTRACT

A system for storing and releasing gases for a breathalyzer test is provided. The gas cartridges and containers of the system can store and release various gaseous mixtures including exhaled air samples. The system can be used for testing breathalyzer devices. These devices include, for example, small hand-held units and larger units that are built into vehicles such as automobiles and trucks. The system is particularly suitable for breath alcohol ignition interlock devices that are used to prevent a driver from operating a motor vehicle when their blood alcohol concentration is at a certain level. In yet another embodiment, the container assembly is used when there is a alcohol sensor for the steering wheel in the vehicle as opposed to a breathalyzer device.

### CROSS-REFERENCE TO RELATED APPLICATIONS

The application claims the benefit under 35 U.S.C. § 371 of PCT International Application No. PCT/US2023/076911 filed Oct. 13, 2023, which claims the benefit of U.S. Provisional Patent Application No. 63/416,077 filed Oct. 14, 2022, the entire disclosures of which are hereby incorporated by reference.

### BACKGROUND OF THE INVENTION

### Field of the Invention

The present invention relates generally to a system for storing and releasing gasses for a breathalyzer test. The system can be used for testing breathalyzer devices, particularly breath alcohol ignition interlock devices that are used to prevent a driver from operating a motor vehicle when their blood alcohol concentration is at a certain level.

### Brief Review of the Related Art

In recent years, various measuring instruments for determining the concentration of alcohol in human breath (hereinafter referred to as “breathalyzer devices”) have been developed. These devices include, for example, small hand-held devices and units that are built into vehicles such as automobiles and trucks. Law enforcement officers routinely use breathalyzer devices on vehicle stops to determine if a driver is intoxicated. Also, many event centers, restaurants and bars have breathalyzer devices that patrons can freely use to check their blood alcohol concentration levels. In general, the breathalyzer device measures the alcohol content of a person's exhaled breath. The breath sample generally has an alcohol content that is directly proportional to the alcohol concentration in the bloodstream.

In particular, breath alcohol ignition interlock devices have been used to prevent a driver from operating a motor vehicle when their blood alcohol concentration is above the legal limit. Typically, the ignition interlock device includes a fuel cell or other type of alcohol sensor, a signal processor, and a display screen. As a driver blows into the breathalyzer device, the alcohol sensor reacts with the alcohol in the exhaled breath and sends an electrical signal that is converted into a blood alcohol concentration value. This alcohol concentration percentage is recorded on the display screen of the device. Such breathalyzer devices are designed to prevent a driver from starting a motor vehicle when the driver's blood alcohol concentration is above a legal limit. If the driver's blood alcohol concentration exceeds a predetermined limit, the vehicle will not start. The breath alcohol ignition interlock device may record this non-starting of the the vehicle as a violation and store this information. On the other hand, if the driver's blood alcohol concentration is below the threshold, the ignition interlock device allows the vehicle to start.

One drawback with such breathalyzer devices is the possibility of false positives being recorded. That is, a person can blow into the breathalyzer device and the device may record that the person has a specific blood alcohol concentration when that person actually does not have that condition. One problem is that even if a person falsely records a positive test result, he/she will likely face significant consequences. A law enforcement officer may arrest and charge the driver with driving under the influence. In order to overcome these charges, the accused person will need to testify in a court hearing. This can be a costly and time-consuming ordeal. The driver also may incur fines and penalties for processing and returning their automobile. Furthermore, the driver may face employment problems if their employer requires a clean record.

Thus, it is important that the breathalyzer device measure the blood alcohol concentration accurately and consistently. When the breathalyzer device is used repeatedly, the fuel cell or other alcohol sensor can become contaminated; and it will not record accurate and consistent test results. Thus, the breathalyzer device needs to be recalibrated periodically. The recalibration process typically involves using a reference gas having a known concentration of alcohol. The reference gas is transmitted to the breathalyzer device; and the indicated output of the device is adjusted to correspond to the known alcohol concentration of the reference gas. There are several references in the patent literature that describe such calibration and recalibration systems as discussed below.

For example, Park, U.S. Patent Application Publications 2014/0358020 and 2015/0297117 disclose methods for calibrating a breathalyzer. The methods in Park '020 and Park '117 involves using gas cartridges containing an alcohol gas with a predetermined concentration of alcohol. The gas cartridge containing the standard alcohol gas is attached to the breathalyzer and the alcohol gas is exhausted into the breathalyzer. Then, the breathalyzer measures the concentration of the standard alcohol gas and compares this value with the predetermined concentration value of the standard alcohol gas. If the actual measured value of the alcohol gas coincides with that of the predetermined concentration value, then there is no need to recalibrate the breathalyzer. If the actual measured value of the alcohol gas does not coincide with that of the predetermined concentration value, then the breathalyzer device needs to be recalibrated.

Jensen et al., U.S. Pat. No. 5,134,875 discloses a breath alcohol simulator and solution containing a known concentration of alcohol. The breath alcohol simulator functions by passing or bubbling air (or an inert gas) through a simulator solution having a fixed fluid concentration of alcohol. The air or gas absorbs a specific molar amount of alcohol molecules and therefore has a known alcohol-to-air concentration. The gas having the known alcohol vapor concentration is fed into a breathalyzer instrument to determine if the instrument needs to be calibrated.

Forrester, U.S. Pat. No. 5,400,637 discloses a system for calibrating a breath alcohol measuring instrument using a gas having a standardized concentration of alcohol. In use, the operator attaches the measuring instrument to a container containing the standard gas and activates the instrument to take a measurement. Immediately before or after measuring the standard, the start button of the instrument is activated, and the expected value is also recorded. If the value of the standard is within 5% of the expected value, the instrument is properly calibrated.

De Vries et al., U.S. Pat. No. 10,877,008 discloses a breath alcohol device calibration system including a computerized calibration module operable to calibrate a breath alcohol device using a reference gas of known alcohol concentration. A reference gas tank identification module is used to identify a reference gas tank that is coupled to the breath alcohol device calibration system. Identification is performed using at least one distinguishing characteristic of the coupled tank,

However, none of the above-discussed references disclose a system, wherein a gas cartridge containing a gaseous mixture of exhaled air with zero alcohol concentration is used for testing a breathalyzer device as found in the present invention. The portable, pressurized container assemblies of the present invention allow a person to use normal exhaled air to test the breathalyzer device. A driver or other person can easily use the container assembly of the invention to test their own condition. The exhaled air gas sample can be prepared so that it does not have any alcohol content and stored in the container assembly. When the air gas sample is charged to the breathalyzer device and reacts with the alcohol sensor, the breathalyzer does not register any alcohol concentration signal. That is, the blood alcohol concentration registers as zero percent (0%); and the person passes the breathalyzer test. The present invention provides additional features, benefits and advantages as described further below.

### SUMMARY OF THE INVENTION

The present invention provides a container assembly for charging a gaseous mixture into a breathalyzer device. In one embodiment, the assembly comprises: a) a main body container having an outer perimeter wall, a top cover, and a bottom base, the cover and base being joined to the outer perimeter wall to define an interior region of the main body container; wherein the top cover includes an upper surface, the upper surface comprising a pin projecting upwardly; an inlet port adapted for introducing a gaseous mixture from a pressurized gas cartridge into the interior region of the main container; and an outlet port adapted for introducing the gaseous mixture from the interior region of the main container into a breathalyzer device; b) a pressurized gas cartridge having an interior region, wherein the gaseous mixture is maintained under pressure, the cartridge having a pierceable membrane so that when the pin of the main container is pushed into the membrane, the gaseous mixture is released from the cartridge and flows into the main container through the inlet port; c) a breathalyzer device adapted for receiving the gaseous mixture from the main container, wherein the gaseous mixture from the interior region of the main container flows into the breathalyzer device through the outlet port.

In another embodiment, the assembly comprises; a) a main body container having an outer perimeter wall, a top cover, and a bottom base, the cover and base being joined to the outer perimeter wall to define an interior region of the main body container; wherein the top cover includes an upper surface, the upper surface comprising an actuating means and an outlet port adapted for introducing the gaseous mixture from the interior region of the main container into a breathalyzer device; and b) a breathalyzer device adapted for receiving the gaseous mixture from the main container when the actuating means is activated.

In yet another embodiment, the container assembly is used when there is an alcohol sensor for the steering wheel in the vehicle, such as an automobile or truck, as opposed to a breathalyzer device.

### BRIEF DESCRIPTION OF THE DRAWINGS

The novel features that are characteristic of the present invention are set forth in the appended claims. However, the preferred embodiments of the invention, together with further objects and attendant advantages, are best understood by reference to the following detailed description in connection with the accompanying drawings in which:

FIG. 1 is a perspective view of one embodiment of the container assembly of the present invention;

FIG. 2 is a perspective view of a second embodiment of the container assembly of the present invention;

FIG. 3 is a perspective view of a third embodiment of the container assembly of the present invention; and

FIG. 4 is a perspective view of a fourth embodiment of the container assembly of the present invention.

### DETAILED DESCRIPTION OF THE INVENTION

The present invention relates generally to a container assembly for storing and releasing gases for a breathalyzer test. The container assembly can be used for testing breathalyzer devices, particularly breath alcohol ignition interlock devices.

### Containers Having Cartridges

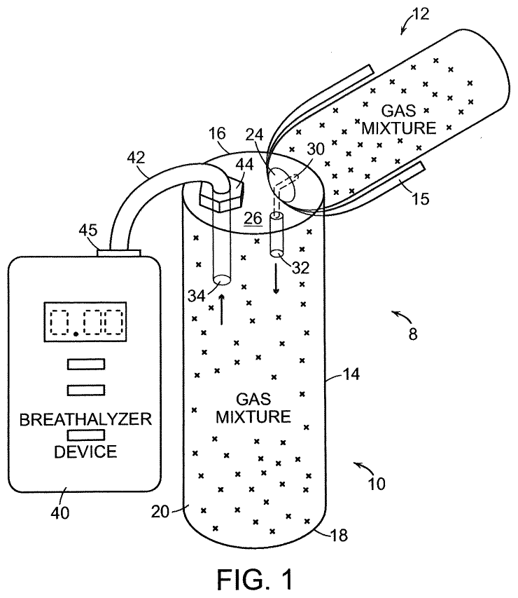

Referring to the Figures, where like reference numerals are used to designate like elements, and particularly FIG. 1, one embodiment of the container assembly of the present invention is shown. The container assembly, generally indicated at (8), comprises a main body container (10). The main container (10) can have any suitable structure, for example, it can have a cylindrical shape as shown in FIG. 1. The main container (10) can be any suitable receptacle including, but not limited to, cans, bottles, vials, vessels, and the like. The main container (10) can be made from metal, plastic, or other suitable material. A pressurized gas cartridge (12) containing a gaseous mixture is coupled to the main body container (10). The composition of the gaseous mixture is described further below.

More particularly, in one preferred embodiment, the main body container (10) includes an outer perimeter wall (14), a top cover (16); and a bottom base (18). The cover (16) and base (18) are joined to the outer perimeter wall (14) to define an interior region or chamber (20) of the main container (10) for holding the gaseous mixture as described further below.

The pressurized gas cartridge (12) is adapted for charging the gaseous mixture into the main body container (10). The pressurized gas cartridge (12) can be supported by a connector frame (15) and coupled to the main container (10) in different ways. In one preferred embodiment, the pressurized gas cartridge (12) contains a soft membrane (24) that is pierceable; and the main container (10) includes an upper surface (26) with a projecting pin (30). The pin (30) can be pushed into the pierceable membrane (24) of the gas cartridge (12), thereby causing the cartridge to puncture. The gas cartridge (12) can be secured tightly to the main container (10) by a twisting or other suitable tightening action. Once punctured, the gaseous mixture inside of the cartridge (12) is released and flows into the interior region (20) of the main container (10). A gas inlet port (32) allows the gaseous mixture to be regulated and fill the interior region (20) of the main container (10). In addition, the main container (10) includes a gas outlet port (34) for delivering the gaseous mixture to a breathalyzer device (40) as described further below. The pressurized gas cartridge (12) also can be any suitable receptacle including, but not limited to, cans, bottles, vials, vessels, and the like. The gas cartridge (12) also can be made from metal, plastic, or other suitable material.

As the gaseous mixture is introduced into the main container (10) through the inlet port (32), it fills the container (10). The gaseous mixture is then discharged from the container (10) through a gas outlet port (34). The main container (10) can be coupled to the breathalyzer device (40) by any suitable means. In one preferred example, the gaseous mixture is delivered from the container (10) through plastic tubing (42) to the breathalyzer device (40). The flow of gas through the gas outlet port (34) and through the plastic tubing (42) can controlled by a metering valve (44). The plastic tubing (42) is connected to the breathalyzer device (40) at breathalyzer inlet port (45). The rate and volume of the gas flowing through the metering valve (44) and through the tubing (42) preferably corresponds to the typical rate and volume of exhaled air by humans. That is, the flow of gas can be directed in a prescribed manner. It should be understood that any suitable metering valve (44) can be used to control the exhaustion of the gas. Such metering valves (44) are known in the art and commercially available. The gas inlet port (32) also can include a metering valve to control the flow of the gaseous mixture from the pressurized gas cartridge (12) and into the main container (10). The assembly (8) may further contain a pressure relief valve to protect the container (10) from over-pressurization and a pressure regulator to control the delivery pressure of the gaseous mixture.

Different breathalyzer devices (40), as known in the art, can be used in accordance with the present invention. These devices include, for example, small hand-held devices and units that are built into vehicles such as automobiles and trucks. Typically, the breathalyzer device (40) includes a fuel cell or other type of alcohol sensor, a signal processor, and a display screen. As a person blows into the breathalyzer device (40), the alcohol sensor reacts with the alcohol in the exhaled breath and sends an electrical signal that is converted into a blood alcohol concentration value. This concentration percentage is recorded on the display screen of the device. In general, a breath sample has an alcohol content that is directly proportion to the alcohol concentration in the bloodstream.

For example, the breathalyzer device (40) can be a breath alcohol ignition interlock device used to prevent a driver from operating a motor vehicle when their blood alcohol concentration is above the legal limit. Such breathalyzer devices are designed to prevent a driver from starting a motor vehicle when the driver's blood alcohol concentration is above a set alcohol concentration. That is, these breathalyzer devices will allow the vehicle to start or run only if certain conditions are met. If the driver's blood alcohol concentration exceeds a predetermined limit, the vehicle will not start. Conversely, if the driver's blood alcohol concentration is below the threshold, the ignition interlock device allows the vehicle to start.

As discussed above, the pressurized gas cartridge (12) of this invention is coupled to the breathalyzer device (40) and the gas inside of the gas cartridge (12) flows into the breathalyzer (40), where it reacts with a fuel cell or other alcohol sensor.

The pressurized gas cartridge (12) can contain different gaseous mixtures in accordance with this invention. In one preferred embodiment, the gas cartridge contains standard exhaled air with a predetermined concentration of the components normally found in exhaled air. For example, the pressurized gas cartridge (12) can comprise a mixture of nitrogen, oxygen, carbon dioxide, argon, water vapor, and other gasses in varying amounts. More particularly, the gaseous mixture can comprise about 4 to about 7% water vapor (preferably about 5 to about 6.3%); about 77 to about 82% nitrogen (preferably, about 79%); about 12 to about 18 % oxygen (preferably about 13.6 to about 16%), about 3 to about 6% carbon dioxide (preferably about 4.0 to about 5.3%); and about 0.5 to about 1.5% argon (preferably about 0.7 to about 1%), which is generally the composition of standard exhaled air.

In the above-described example, where the gas sample is standard exhaled air, the sample does not contain any alcohol parts. Thus, as the gas sample flows into the breathalyzer device and reacts with the alcohol sensor, the device should not register any alcohol concentration signal. That is, the blood alcohol concentration should register as zero percent (0%); and the person should pass the breathalyzer test. If the breathalyzer device (40) is a breath alcohol ignition interlock device as described above, the device should allow the vehicle to start because the alcohol content of the gas (air) sample is zero.

It is important that the breathalyzer device (40) measure the blood alcohol concentration accurately and consistently. As described above, the gas sample, which is charged into the breathalyzer device, can be standard exhaled air with zero alcohol content. In such an example, the breathalyzer device (40) should record a breath alcohol level of zero percent (0%). If the breathalyzer device (40) does not record an alcohol concentration value of zero percent, then it would be clear the device was not operating properly.

Although the container assembly is described primarily herein as using standard exhaled air as the gas sample, it should be understood that any suitable gas can be used. For example, in another embodiment, the gas sample can have some alcohol content. For example, a gaseous mixture comprising about 4 to about 7% water vapor; about 77 to about 82% nitrogen; about 12 to about 18% oxygen; about 3 to about 6% carbon dioxide; about 0.5 to about 1.5% argon; and about 0.01 to about 0.10% alcohol can be used.

### Containers Having No Cartridges

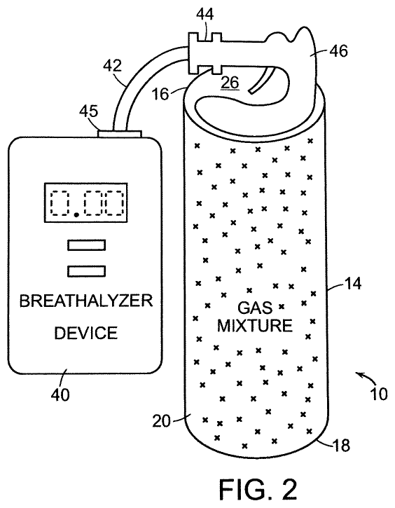

Turning to FIG. 2, in another embodiment, a pressurized gas cartridge (12) containing the stored gaseous mixture sample is not used. Rather, the gas sample is stored directly in the interior region or chamber (20) of the main body container (10). The main container (10) can contain any suitable actuating means that can function to release the gaseous mixture from the container. For example, the actuator can be a trigger-activated dispenser (46) having a trigger that is pulled to open the metered valve (44) and discharge the gas sample to the breathalyzer device (40). Once the trigger is pulled on the trigger-activated dispenser (46), the stored gas sample inside of the main container (10) is delivered to the breathalyzer device (40). As described above, the main container (10) can be coupled to the breathalyzer device (40) by any suitable means such as, for example, plastic tubing (42). It should be understood that other actuator elements, for example, buttons, knobs, and the like can be used to activate the main container (10) and release the gas sample. In turn, the gas sample is fed from the main container (10) to the breathalyzer device (40). In a manner similar to the container assembly (8) described above, the flow of gas to the breathalyzer device (40) can be controlled by a metered valve (44). The flow of gas to the breathalyzer (40) can be controlled so that it preferably corresponds to the typical rate and volume of exhaled air by humans.

### Containers For Use With Alcohol Sensors in the Steering Wheel

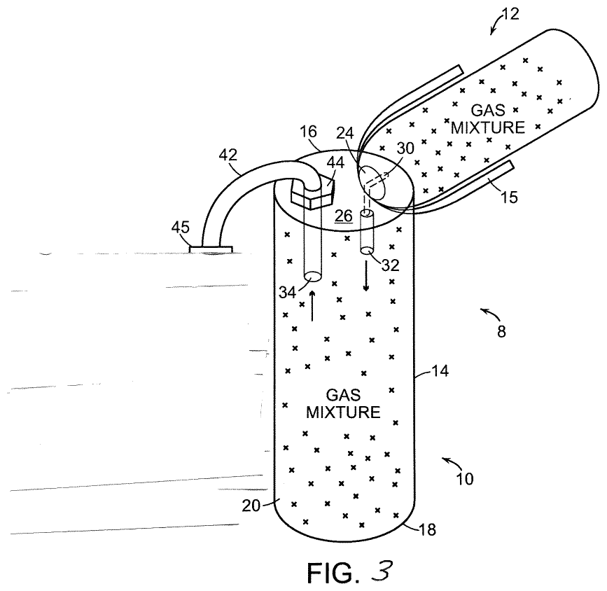

In FIG. 3, another embodiment of the container assembly of the present invention is shown. In this embodiment, the container assembly is used when there is an alcohol sensor for the steering wheel in the vehicle as opposed to a breathalyzer device. The container assembly, generally indicated at (8), comprises a main body container (10). The main container (10) can have any suitable structure; for example, it can have a cylindrical shape as shown in FIG. 3.

As shown in FIG. 3, the main body container (10) includes an outer perimeter wall (14), a top cover (16); and a bottom base (18). The cover (16) and base (18) are joined to the outer perimeter wall (14) to define an interior region or chamber (20) of the main container (10) for holding the gaseous mixture as described further below.

The pressurized gas cartridge (12) is adapted for charging the gaseous mixture into the main body container (10). The pressurized gas cartridge (12) can be supported by a connector frame (15) and coupled to the main container (10) in different ways. In one preferred embodiment, the pressurized gas cartridge (12) contains a soft membrane (24) that is pierceable; and the main container (10) includes an upper surface (26) with a projecting pin (30). The pin (30) can be pushed into the pierceable membrane (24) of the gas cartridge (12), thereby causing the cartridge to puncture. The gas cartridge (12) can be secured tightly to the main container (10) by a twisting or other suitable tightening action. Once punctured, the gaseous mixture inside of the cartridge (12) is released and flows into the interior region (20) of the main container (10). A gas inlet port (32) allows the gaseous mixture to be regulated and fill the interior region (20) of the main container (10). In addition, the main container (10) includes a gas outlet port (34) for delivering the gaseous mixture to a breathalyzer device (40) as described further below. The pressurized gas cartridge (12) also can be any suitable receptacle including, but not limited to, cans, bottles, vials, vessels, and the like. The gas cartridge (12) also can be made from metal, plastic, or other suitable material.

As the gaseous mixture is introduced into the main container (10) through the inlet port (32), it fills the container (10). The gaseous mixture is then discharged from the container (10) through a gas outlet port (34). As opposed to FIG. 1, the main container (10) in the embodiment of FIG. 3 is not coupled to a breathalyzer device. Rather, the outlet port (34) and plastic tubing (42) are detachable and can be used when there is an alcohol sensor in the steering wheel. In one preferred example, the gaseous mixture is delivered from the container (10) through plastic tubing (42). The flow of gas through the gas outlet port (34) and through the plastic tubing (42) can be controlled by a metering valve (44). The rate and volume of the gas flowing through the metering valve (44) and through the tubing (42) preferably corresponds to the typical rate and volume of exhaled air by humans. That is, the flow of gas can be directed in a prescribed manner. It should be understood that any suitable metering valve (44) can be used to control the exhaustion of the gas. Such metering valves (44) are known in the art and commercially available. The gas inlet port (32) also can include a metering valve to control the flow of the gaseous mixture from the pressurized gas cartridge (12) and into the main container (10). The assembly (8) may further contain a pressure relief valve to protect the container (10) from over-pressurization and a pressure regulator to control the delivery pressure of the gaseous mixture.

The pressurized gas cartridge (12) can contain different gaseous mixtures in accordance with this invention. In one preferred embodiment, the gas cartridge contains standard exhaled air with a predetermined concentration of the components normally found in exhaled air. For example, the pressurized gas cartridge (12) can comprise a mixture of nitrogen, oxygen, carbon dioxide, argon, water vapor, and other gases in varying amounts. More particularly, the gaseous mixture can comprise about 4 to about 7% water vapor (preferably about 5 to about 6.3%); about 77 to about 82% nitrogen (preferably, about 78%); about 12 to about 18 % oxygen (preferably about 16%), about 3 to about 6% carbon dioxide (preferably about 5%); and about 0.5 to about 1.5% argon (preferably about 1%), which is generally the composition of standard exhaled air.

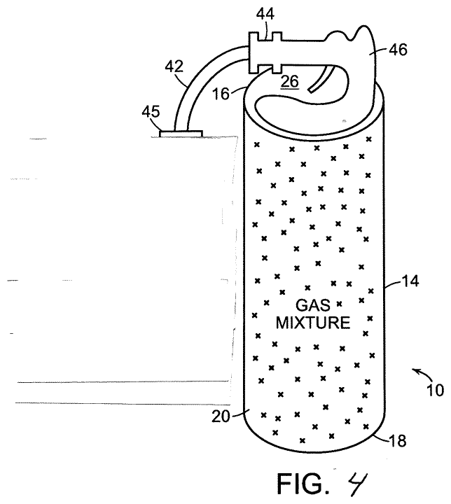

In yet another embodiment, as shown in FIG. 4, a pressurized gas cartridge (12) containing the stored gaseous mixture sample is not used. Rather, in a manner similar to the container assembly in FIG. 3, the container assembly in FIG. 4 is used when there is an alcohol sensor for the steering wheel in the vehicle as opposed to a breathalyzer device. In the embodiment of FIG. 4, the gas sample is stored directly in the interior region or chamber (20) of the main body container (10). The main container (10) can contain any suitable actuating means that can function to release the gaseous mixture from the container. For example, the actuator can be a trigger-activated dispenser (46) having a trigger that is pulled to open the metered valve (44) and discharge the gas sample. Once the trigger is pulled on the trigger-activated dispenser (46), the stored gas sample inside of the main container (10) is delivered through the plastic tubing (42). The plastic tubing (42) are detachable and can be used when there is an alcohol sensor in the steering wheel. It should be understood that other actuator elements, for example, buttons, knobs, and the like can be used to activate the main container (10) and release the gas sample. In a manner similar to the container assembly (8) described above, the flow of gas can be controlled by a metered valve (44). The flow of gas can be controlled so that it preferably corresponds to the typical rate and volume of exhaled air by humans.

It should be understood the terms, “first”, “second”, “third”, “top”, “bottom”. “inner”, “outer”, “below”, “above”. “upper”, “lower”, “upward”, “downward”, “right”, “left”, “anterior”, “posterior”, and the like are arbitrary terms used to refer to one position of an element based on one perspective and should not be construed as limiting the scope of the invention.

It also should be understood that the assemblies, constructions, materials, methods, and the like described and illustrated herein represent only some embodiments of the invention. It is appreciated by those skilled in the art that various changes and additions can be made to the assemblies, systems, components, constructions, materials, methods, and the like without departing from the spirit and scope of this invention. It is intended that all such embodiments be covered by the appended claims.

I claim:

1. A container assembly for charging a gaseous mixture into a breathalyzer device, the assembly comprising;
a) a main body container having an outer perimeter wall, a top cover, and a bottom base, the cover and base being joined to the outer perimeter wall to define an interior region of the main body container;
wherein the top cover includes an upper surface, the upper surface comprising a pin projecting upwardly; an inlet port adapted for introducing a gaseous mixture from a pressurized gas cartridge into the interior region of the main container; and an outlet port adapted for introducing the gaseous mixture from the interior region of the main container into a breathalyzer device;
b) a pressurized gas cartridge having an interior region, wherein the gaseous mixture is maintained under pressure, the cartridge having a pierceable membrane so that when the pin of the main container is pushed into the membrane, the gaseous mixture is released from the cartridge and flows into the main container through the inlet port; and
c) a breathalyzer device adapted for receiving the gaseous mixture from the main container, wherein the gaseous mixture from the interior region of the main container flows into the breathalyzer device through the outlet port.

2. The container assembly of claim 1, wherein the main container has cylindrical structure.

3. The container assembly of claim 1, wherein the outlet port comprises a metered valve for introducing the gaseous mixture into the breathalyzer device.

4. The container assembly of claim 1, wherein the main container is coupled to the breathalyzer device by plastic tubing.

5. The container assembly of claim 1, wherein the breathalyzer device is a breath alcohol ignition interlock system.

6. The container assembly of claim 1, wherein the gaseous mixture comprises nitrogen, oxygen, carbon dioxide, argon, and water vapor.

7. The container assembly of claim 6, wherein the gaseous mixture comprises about 4 to about 7% water vapor; about 12 to about 18 % oxygen; about 3 to about 6% carbon dioxide; and about 0.5 to about 1.5% argon.

8. The container assembly of claim 6, wherein the gaseous mixture comprises about 4 to about 7% water vapor; about 12 to about 18 % oxygen; about 3 to about 6% carbon dioxide; and about 0.5 to about 1.5% argon; and about 0.01 to about 0.10% alcohol.

9. A container assembly for charging a gaseous mixture into a breathalyzer device, the assembly comprising;
a) a main body container having an outer perimeter wall, a top cover, and a bottom base, the cover and base being joined to the outer perimeter wall to define an interior region of the main body container;
wherein the top cover includes an upper surface, the upper surface comprising an actuating means and an outlet port adapted for introducing the gaseous mixture from the interior region of the main container into a breathalyzer device; and
b) a breathalyzer device adapted for receiving the gaseous mixture from the main container when the actuating means is activated.

10. The container assembly of claim 9, wherein the actuating means is a trigger-activated dispenser that is pulled to introduce the gaseous mixture into the breathalyzer device.

11. The container assembly of claim 9, wherein the outlet port comprises a metered valve for introducing the gaseous mixture into the breathalyzer device.

12. The container assembly of claim 9, wherein the main container is coupled to the breathalyzer device by plastic tubing.

13. The container assembly of claim 9, wherein the breathalyzer device is a breath alcohol ignition interlock system.

14. The container assembly of claim 9, wherein the gaseous mixture comprises nitrogen, oxygen, carbon dioxide, argon, and water vapor.

15. The container assembly of claim 14, wherein the gaseous mixture comprises about 4 to about 7% water vapor; about 12 to about 18 % oxygen; about 3 to about 6% carbon dioxide; and about 0.5 to about 1.5% argon.
